(19) United States
(12) Patent Application Publication
(10) Pub. No.: US 2009/0254187 A1
(43) Pub. Date: Oct. 8, 2009

(54) Tightenable Surgical Retractor Joint

(75) Inventor: Todd M. BJORK, River Falls, WI (US)

Correspondence Address: SHEWCHUK IP SERVICES, 3356 SHERMAN CT. STE. 102, EAGAN, MN, 55121, US

(73) Assignee: MINNESOTA SCIENTIFIC, INC., St. Paul, MN (US)

(21) Appl. No.: 12/099,125

(22) Filed: Apr. 7, 2008

(51) Int. Cl. A61F 2/30; A61B 1/32; A61F 2/40

(52) U.S. Cl. 623/18.11; 600/201; 623/19.12

(57) ABSTRACT

A tightenable joint for use in a surgical retractor system includes a monolithic, unitary joint body which has a top leg and a bottom leg. The top leg provides a top compression surface and the bottom leg provides a bottom compression surface operating on a ball. A handled cam is used between proximal ends of the top leg and the bottom leg. When the handle is thrown to tighten the joint, the proximal ends of the top leg and the bottom leg are driven apart by the cam, and a hinge or fulcrum portion on the joint body flexes between top and bottom leg portion such that the top compression surface and bottom compression surface tighten on the ball. The ball has flats that enable the ball to be positioned into the socket, and then an extension is fixed to the ball to prevent removal of the ball from the socket. The cam is defined with an eccentric surface relative to its pivot pin, which is received in an arcuate recess of a bearing plate. When the handle is thrown, the bearing plate slides relative to the joint body under the cam.

### CROSS-REFERENCE TO RELATED APPLICATION(S)

None.

### BACKGROUND OF THE INVENTION

The present invention relates to the field of surgical tools, and particularly to the design and manufacture of surgical retractor systems. Surgical retractor systems have long been used during surgery to bias and hold tissue in a desired position. In many retractor systems, clamps and joints are used which have a loosened position in which the post, shaft, retractor blade and/or other portions of the assembly can be easily moved, and a tightened position in which the connection is held rigid.

Numerous such surgical retractor clamps and joints exist in the prior art. Many surgical retractor clamps operate on bar stock of other components of the surgical retractor systems. In some surgical retractor systems the bar stock is generally cylindrical, and the cylindrical bar stock can be positioned at incrementally different positions relative to the clamp by rotating the bar stock about its longitudinal axis (or vice versa). In other surgical retractor systems including systems known as “Bookwalter/Codman” systems, the bar stock is rectangular in cross-section or otherwise has longitudinally extending flatted sides. The flatted sides of the bar stock generally prevent any rotational movement or slippage of the bar stock about its longitudinal axis relative to the clamp, but also eliminated the possibility of fine adjustment of the circumferential position of the flats.

Ball-and-socket joints are well known in a wide variety of fields including the surgical retractor field. Ball-and-socket joints include a generally spherical ball having a ball shaft extending off one side. The ball is placed in a socket that while loosened holds the center of the ball in a set position but permits pivoting, commonly allowing simultaneous pivoting adjustment in pitch, in yaw and in roll. Many ball-and-socket joints can then be tightened to prevent any movement of the ball and ball shaft relative to the socket. If desired, a track or interference structure can be added to ball-and-socket joint to limit or restrict motion in one or more of pitch, yaw and roll, i.e., so the ball is not free to pivot in all directions even while the joint is loosened. However, because ball-and-socket joints generally provide three degrees of freedom (pitch, yaw and roll), they are particularly beneficial when used with flatted bar stock to provide the circumferential adjustment which is otherwise restricted by the flats.

In some ball-and-socket joints, the dimensions of the socket are essentially fixed, and the tightening action involves pushing or pulling the ball against one side of the socket. As one example, the socket may be formed with a conical profile, perhaps with a flexible bushing between the ball and the conical socket. By advancing the ball relative to the conical socket, the frictional force between the socket and the ball increases, tightening the ball-and-socket joint. In some designs, the force advancing the ball relative to the conical socket is provided by a cable axially threaded through a hole in the ball and in the conical socket.

In other ball-and-socket joints, the dimensions of the socket change. For instance, the socket may be made up of two different components which are hinged or otherwise moveable relative to each other, with a tightening mechanism that closes the hinged socket portions around the ball. For instance, U.S. Pat. No. 6,602,190, owned by the assignee of the present invention, discloses a multi-position spherical retractor holder which uses a retaining member tightened against the Bookwalter/Codman frame. In other designs, the socket is formed by a hoop around the ball, and tightening of the hoop and increasing hoop stress makes the hoop smaller and increases friction between the hoop and the ball. For instance, U.S. Pat. Nos. 5,899,627 and 6,264,396, owned by the assignee of the present invention, discloses a ball socket clamping device using a hoop stress to tighten around two balls, one on each end of a cylindrical hoop.

Surgical retractor systems must be robust and strong, as even a possibility of failure during use is not tolerated. Surgical retractor assemblies should be readily reusable, including sterilizable, for use in multiple surgeries. Surgical retractor systems should maintain a relatively low cost, including utilizing as few and simple parts as possible during the manufacturing process. Surgical retractor systems should be easy to use, including as few assembled parts as necessary to minimize assembly time and the possibility of one part being misplaced during use prior to assembly, while still permitting adequate flexibility for the surgeon to perform the desired retraction. The handle or other tightening control should be readily accessible for use, but unobtrusive during the surgical operation. Improvements in surgical retractor systems can be made in keeping with these goals.

### BRIEF SUMMARY OF THE INVENTION

The present invention is a tightenable joint for use in a surgical retractor system. In one aspect, a monolithic, unitary joint body provides both a top compression surface and a bottom compression surface operating on a ball. A hinge or fulcrum portion on the joint body flexes between top and bottom leg portions, and a tightening mechanism operates to spread proximal ends of the legs so the top compression surface and bottom compression surface tighten on the ball. In another aspect, the ball has flats that enable the ball to be positioned into the socket, and then an extension is fixed to the ball to prevent removal of the ball from the socket. In another aspect, the tightening mechanism for the joint includes a cam received in arcuate recesses of a bearing plate, and the bearing plate slides relative to the joint structure during the throw of the handle.

### BRIEF DESCRIPTION OF THE DRAWINGS

FIG. 1 is a perspective view of a surgical retractor clamp utilizing a ball-and-socket joint in accordance with a preferred embodiment of the present invention, shown in a loosened position, with the shaft guide subassembly positioned upright.

FIG. 2 is an exploded perspective view of the component parts in the surgical retractor clamp of FIG. 1.

FIG. 3 is a side view of the joint body of the surgical retractor clamp of FIGS. 1 and 2.

FIG. 4 is a top view of the joint body of the surgical retractor clamp of FIGS. 1 and 2.

FIG. 5 is a front view of the joint body of the surgical retractor clamp of FIGS. 1 and 2.

While the above-identified drawing figures set one or more forth preferred embodiments, other embodiments of the present invention are also contemplated, some of which are noted in the discussion. In all cases, this disclosure presents the illustrated embodiments of the present invention by way of representation and not limitation. Numerous other minor modifications and embodiments can be devised by those skilled in the art which fall within the scope and spirit of the principles of this invention.

### DETAILED DESCRIPTION

A surgical retractor clamp 10 representing a preferred embodiment of the present invention includes five primary components: a joint body 12 which provides a socket 14, a shaft guide subassembly 16 which includes a ball 18, a jaw 20, a sliding bearing plate 22 and an actuating handle 24. The handle 24 is pivotably connected to the joint body 12 by a handle pivot pin 26. The jaw 20 is pivotably connected to the joint body 12 with a jaw pin 28. A compression spring 30 is housed between the jaw 20 and the joint body 12. After the ball 18 has been positioned in the socket 14, a locking pin 32 is fixed to the ball 18 to prevent removal of the ball 18 from the socket 14.

The preferred surgical retractor clamp 10 is designed for use in ways similar to the multi-position spherical retractor holder of U.S. Pat. No. 6,602,190, owned by the assignee of the present invention and incorporated by reference. That is, the preferred surgical retractor clamp 10 is designed for holding components of existing Bookwalter/Codman type systems, such as those components shown in the various patents of John R. Bookwalter et al. including U.S. Pat. Nos. 4,254,763, 4,421,108, 4,424,724, 4,467,791, 5,375,481, 5,520,608, 6,241,659, 6,530,882 and 6,808,493, all incorporated by reference, as originally made and marketed by Codman & Shurtleff, Inc. of Randolph, Mass. Additional examples include those shown in U.S. Pat. Nos. 1,919,120, 1,963,173, 4,434,791, and 5,520,610, all incorporated by reference. In general terms, the Bookwalter/Codman components include retractor blades (not shown) having square/notched retractor blade shafts which extend rearwardly from the surgical arena from the top center of downwardly extending blades. The retractor blades are mounted on an oval or horseshoe shaped frame (not shown) of stock which is rectangular in cross-section, often having crescent-shaped notches around the outside of the frame.

Being designed to hold and tension existing Bookwalter/Codman blade shafts, the preferred shaft guide subassembly 16 operates as known in the art with a spring loaded pawl 34 mounted on a shaft guide receptacle 36 with a pivot pin 38. The shaft guide receptacle 36 includes a square or nearly square passageway 40 therethrough roughly matching the cross-section of existing Bookwalter/Codman blade shafts. Under a light spring force, the pawl 34 clicks into the notches of the Bookwalter/Codman blade shaft. The pawl 34 includes a thumb button 42 which can be pressed to remove the pawl 34 from the notches of the Bookwalter/Codman blade shaft, allowing the Bookwalter/Codman blade shaft to be freely slid forward or rearward in the passageway 40. While the preferred the shaft guide subassembly 16 is designed for clamping Bookwalter/Codman blade shafts, it could be readily modified into any type of clamping or surgical retractor component and still use the ball-and-socket joint of the present invention.

The ball 18 of the preferred embodiment is generally spherical. With a spherical ball 18, the ball 18 is received in the socket 14 with degrees of freedom in pitch, in roll and in yaw. The shaft guide subassembly 16 can pivot in any direction with the center of the ball 18 at the center of the socket 14, until the ball shaft contacts the edge of the socket 14.

The socket 14 which receives the ball 18 is provided in the monolithic joint body 12, which singly provides both a top compression surface 44 and a bottom compression surface 46 around a hinge or fulcrum location 48. Throughout this specification, the terms “top”, “bottom” and similar directional terms are applied based upon the orientation of the clamp 10 shown in the figures; though most commonly used in this orientation, the clamp 10 can be used in any orientation, including being flipped over so the top compression surface 44 is lower in elevation than the bottom compression surface 46. Both the top compression surface 44 and the bottom compression surface 46 are defined with spherical profiles that generally match the radius of the ball 18. In particular, in the loosened state, the top compression surface 44 and the bottom compression surface 46 should define a generally spherical recess which is slightly larger than the outer diameter of the ball 18. The top compression surface 44 and the bottom compression surface 46 should wrap around the ball 18 at least enough to circumscribe 180° of the ball 18, so tightening of the joint 10 can involve equal and opposite forces between the top compression surface 44 and the bottom compression surface 46 on opposing faces of the ball 18. Separate from the torque forces to be withstood by the clamp 10, the wrap of the top compression surface 44 and bottom compression surface 46 around the ball 18 needs to be sufficient to withstand the pull forces to which the clamp 10 will be subjected. The distal ends of the top compression surface 44 and the bottom compression surface 46 thus act as capture extensions to retain the ball 18 in the socket 14. Because the clamp 10 is primarily intended to be used in-line with the pull force (i.e., the pull force during retraction use of the clamp 10 is oriented to pull the ball 18 out of the socket 14), a significant amount of wrapping by the capture extensions is necessary, such as between about 190° and 270°. In the preferred embodiment, the top and bottom compression surfaces 44, 46 provide a wrap angle α of about 220° to prevent the ball 18 from pulling out of the joint body 12. Other designs, having a wrap angle α less than 190°, could allow the ball 14 to snap in and out of the socket 18 when the joint 10 is loosened.

With this large amount of wrap angle α and using a monolithic joint body 12, there needs to be a way of inserting the ball 18 into the socket 14 during assembly of the joint 10. The preferred method of assembly involves two flats 50 which are located on opposing side surfaces of the ball 18. With the 220° wrap angle α, the assembly opening 52 is about 94% of the diameter of the ball 18. Thus, the flats 50 should be slightly less than this 94% of the diameter of the ball 18. With the flats 50 aligned relative to the wrap angle α, the ball 18 can be inserted into the socket 14. Once fully inserted into the socket 14, the ball 18 can be roll rotated to present the flats 50 on the sides of the socket 14.

The right and left sides of the socket 14 must also wrap to some extent around the ball 18 so the ball 18 does not translate to the right or left relative to the socket 14, such as between about 190° and 270° in the side-to-side direction. The amount of the side-to-side wrap depends upon the likely sideways pull out forces of the ball 18 from the socket 14, which is weighed against the range of motion of the joint 10 which is lost if the socket 14 is designed to wrap further around the ball 18. In the preferred embodiment, the top and bottom compression surfaces 44, 46 each provide a spherical contact. The top compression surface 44 wraps about 70° around the ball 18 in the side-to-side direction, and the bottom compression surface 46 wraps about 66° around the ball 18 in the side-to-side direction. Thus, the wrap from the right edge of the top compression surface 44 around the ball 18 to the right edge of the bottom compression surface 46 is about 248° against pull out to the right, and the wrap from the left edge of the top compression surface 44 around the ball 18 to the left edge of the bottom compression surface 46 is about 248° against pull out to the left. Depending upon the range or motion desired for the joint 10, either the amount of top side-to-side wrap or the amount of bottom side-to-side wrap could be adjusted, including providing the vast majority of side-to-side wrap on only one of the top compression surface 44 or bottom compression surface 46.

In the most preferred embodiment, the top compression surface 44 is provided by a top right compression area 44r and a top left compression area 44l, and the bottom compression surface 46 is provided by a bottom right compression area 46r and a bottom left compression area 46l. A central recess 54 splits the right compression areas 44r, 46r from the left compression areas 44l, 46l. Each compression area 44r, 44l, 46r, 46l has a spherical profile for contact with the ball 18. By providing four separated compression areas 44r, 44l, 46r, 46l, tolerances on the ball 18 and tolerances on the joint body 12 are less critical; that is, the ball 18 more easily positions itself centered in the socket 14 without binding or toggling during tightening, even if the shape, size or position of the ball 18 does not perfectly match the shape, size or position of the socket 14.

After the ball 18 is inserted into the socket 14, a ball lock pin 32 is fixed to the ball 18. The ball lock pin 32 could be screw threaded or otherwise removably fixed to the ball 18, or it can be permanently fixed to the ball 18. The preferred ball lock pin 32 is longer than the distance between the flats 50 and extends all the way through the ball 18, such that the addition of the lock pin 32 increases the effective radius of the ball 18 at the flats 50, thereafter preventing the ball 18 from pulling through the assembly opening 52. If desired, the ball lock pin 32 can be made as long as or shorter than the diameter of the ball 18, such that the lock pin 32 does not impede the motion of the joint 10. For instance, one embodiment (not shown) uses a lock pin hole which has its diameter the full size of the flats, i.e., the entirety of the flats is provided by the lock pin hole. A wide diameter lock pin fills the lock pin hole with ends sized and contoured to match the spherical profile of the ball 18. Alternatively and as shown in the drawings, the ball lock pin 32 can be made longer than the diameter of the ball 18, or otherwise attached to the ball 18 such that the ball lock pin 32 extends beyond the spherical profile and interferes with the socket 14. With an interfering lock pin, the lock pin 32 prevents full roll rotation of the ball 18 and prevents any alignment of the flats 50 with the assembly opening 52. With a non-interfering lock pin (not shown), the ball 18 can be fully roll rotated so the flats 50 line up with the assembly opening 52, in which case the entirety the ball 18 may be able to pull slightly forward with the entirety of a pull-out force bourn by the lock pin.

The socket 14 has side openings 56 sized to permit insertion of the ball lock pin 32 into the ball lock pin hole 58 while the ball 18 is in the socket 14. In the preferred embodiment, the open sides 56 of the socket 14 progress back toward the fulcrum 48. The open sides 56 permit a wider range of side-to-side (yaw) motion of the joint 10 before the interfering lock pin 32 contacts the edge of the socket 14. As importantly, the fulcrum location and size control the amount and direction of deflection of the top compression surface 44 relative to the bottom compression surface 46 during tightening of the clamp 10.

The diameter of the ball 18 is selected based primarily upon the required torque and pull forces that the joint 10 has to withstand during use. The frictional torque imposed on the ball 18 by the top and bottom compression surfaces 44, 46 is a function of the compression force placed on the ball 18 by the top and bottom compression surfaces 44, 46 multiplied by the ball diameter, so a larger diameter ball 18 results in a joint 10 which can withstand more pitch, yaw and roll forces. On the other hand, a smaller joint is preferred to provide a smaller, less obtrusive profile to the entire clamp 10, so the joint can be made as small as possible so long as it can provide sufficient clamping force. In the preferred embodiment, the ball 18 has an outer diameter of about ⅝ inches. In the preferred embodiment, the top compression surface 44 and the bottom compression surface 46 in the loosened configuration define an inner diameter slightly larger than the outer diameter of the ball 18, such as about 0.01 inches greater in diameter. This 0.01 clearance provides essentially friction free rotational movement but defined position of ball 18 during loosened positioning of the joint 10. The ball shaft 42 is as large as necessary to transmit the expected forces, but otherwise a smaller ball shaft leads to greater angles of motion for the joint 10. With the ⅝ inch diameter ball 18, a ¼ inch diameter ball shaft 42 is appropriate. The flats 50 of the preferred embodiment are about 0.54 inches apart, i.e., each flat shaves about 0.04 inches off the curvature of the spherical ball 18. At this size, each flat is a circle of about 0.3 inches in diameter. The lock pin hole 58 and the ball lock pin 32 in the preferred embodiment are considerably smaller in diameter than the flats 50, such as a diameter of about ⅛ inch.

With this size of ball 18, ball shaft 42 and socket profile, the ball 18, ball shaft 42 and receptacle 36 can pivot upward and downward through a total pitch angle θ of about 92°. For the desired orientation of a blade shaft through the blade shaft receptacle 36 for desired retraction forces, the pitch angle is split into a maximum declining pitch angle θ_{d} of about 0° before the ball shaft 42 contacts the bottom compression surface 46 and a maximum inclining pitch angle θᵢ of about 92° before the ball shaft 42 38 contacts the top compression surface 44. The joint 10 can be designed with greater or lesser maximum declining pitch angle θ_{d} and maximum inclining pitch angle θᵢ depending upon the amount of freedom required of the joint 10.

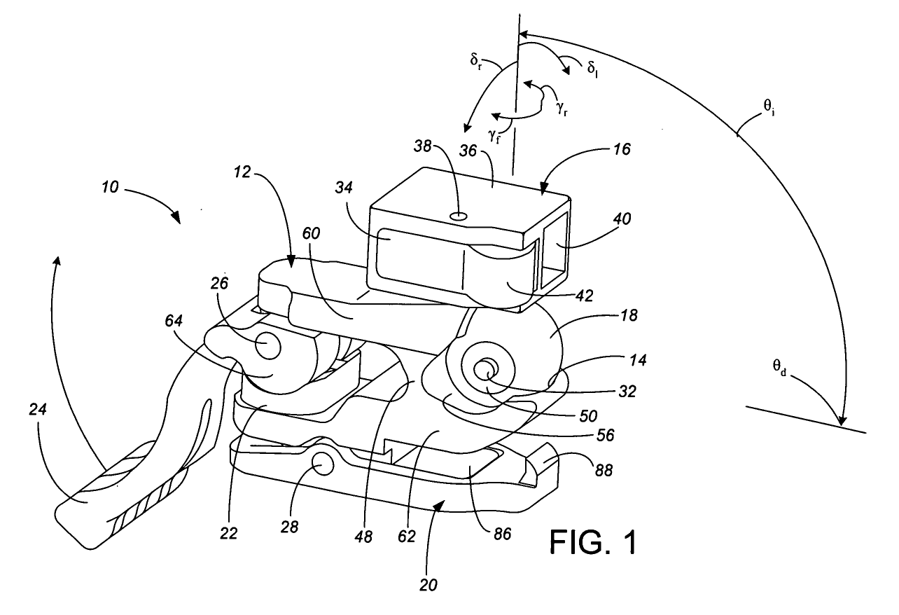

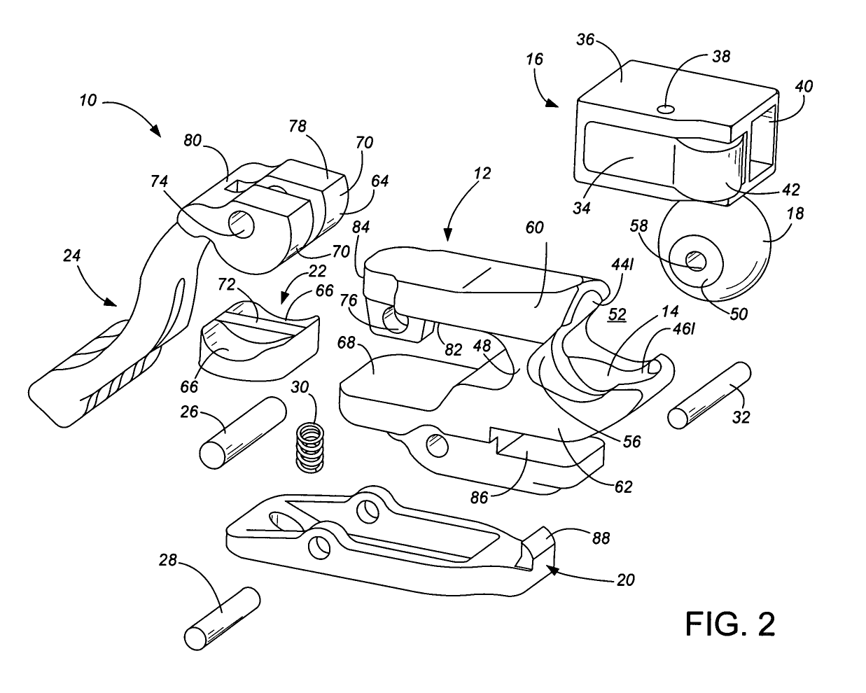

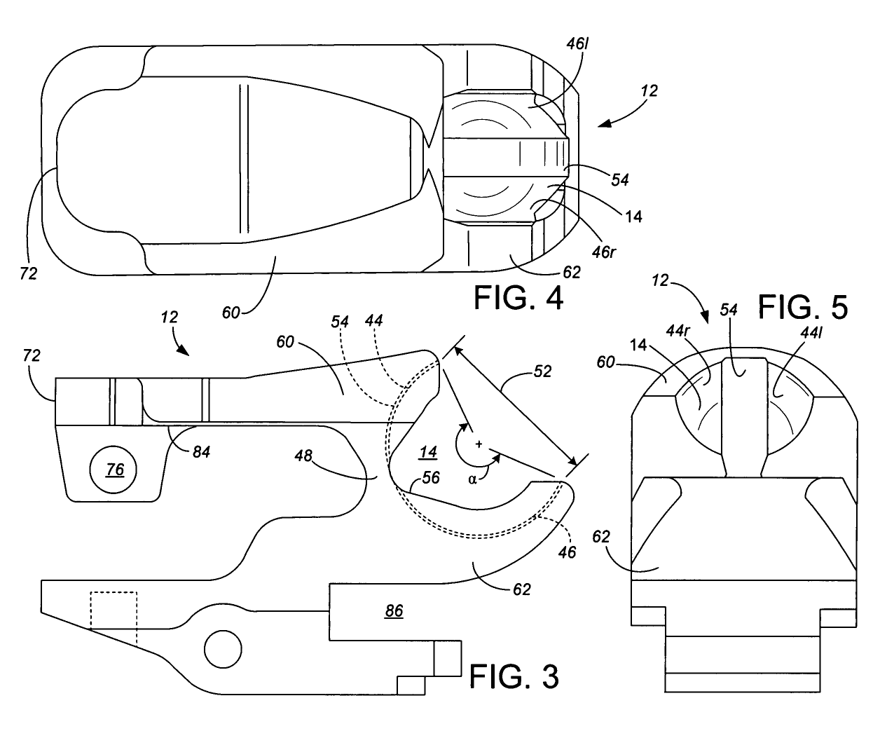

The relative size of the ball lock pin 32 as compared to the side openings 56 permits the ball 18, ball shaft 42 and receptacle 36 to twist about the ball shaft axis 44 through a left twist (yaw) angle δₗ and through a right twist (yaw) angle δᵣ. The relative size of the ball lock pin 32 as compared to the side openings 56 also permits the ball 18, ball shaft 42 and receptacle 36 to pivot forward and backward through maximum roll angles γ_{f} and γ_{b}. With the preferred ⅛ inch diameter interfering ball lock pin 32, the preferred joint body 12 uses side openings 56 of about 7/16 inches. The deep position of the fulcrum 48 permits maximum roll angles γ_{f} and γ_{b} both clockwise and counterclockwise of about 45° while at the upright position shown in FIG. 1. The side openings 56 allow maximum yaw angles δₗ and δᵣ both to the right and the left of about 30° while at the upright position shown in FIG. 1. Modifications to the relative shapes and sizes of the lock pin 32 and/or ball shaft and side openings 56 can permit wide variations to these angles, as desired for the degree of joint flexibility needed in any particular application.

Though the joint body 12 is formed as a unitary structure, the fulcrum or hinge area 48 provides an area of bending flexibility such that during tightening of the joint 10 the top portion 60 of the joint body 12 slightly rotates relative to the bottom portion 62 of the joint body 12 by bending at the fulcrum 48. Thus, the joint body 12 in function operates in some ways similarly to the fulcrum clamps of U.S. Pat. Nos. 5,727,899, 7,297,107 and 7,320,666, owned by the assignee of the present invention and incorporated by reference.

The top compression area 44 and the bottom compression area 46 are each only about half an inch from the fulcrum 48, while the pivot pin 26 for the handle 24 is about 0.9 inches from the fulcrum 48. The handle 24 operates a cam 64 positioned between the handle pivot pin 26 and the bearing plate 22, such that the cam 64 pushes against longer lever arms to the fulcrum 48 than the lever arms of the top and bottom compression areas 44, 46. The curvature of the cam 64 is eccentric relative to the pivot pin 26; in the preferred embodiment, the effective radius of the cam 64 changes about 0.06 inches over a throw of about 90°. Due to the relative length of lever arms about the fulcrum 48, when the ball 18 is not in the socket 14, the throw of the handle 24 results in the top compression area 44 moving about 0.03 inches closer to the bottom compression area 46. With the ball 18 in the socket 14, the first third of the handle throw is taken up in absorbing the 0.01 clearance between the ball 18 and the socket 14, and the second two thirds of the handle throw is taken up as a very strong compression force on the ball 18.

The cam 64 does not bear against a flat surface, but rather bears against the curved surface 66 of the bearing plate 22. The curvature of the bearing surface 66 almost exactly matches the curvature of the cam 64, with the preferred embodiment having a 0.251 inch inner cylindrical radius of the bearing plate 22 bearing against a 0.250 inch outer cylindrical radius of the cam 64. With a curved inner radius of the bearing plate 22, the bearing force is spread across a considerably larger contact area than if the cam 64 bore against a planar surface. The preferred bearing plate 22 is formed of a lubricious polymer material such as PEEK, to minimize the friction between the bearing plate 22 and the cam 64. Other than the bearing plate 22, the other components of the joint 10, including particularly the cam 64 of the handle 24 and the planar sliding surface 68 of the lower leg 62, can be formed of surgical grade stainless steel.

With the inner surface 66 of the bearing plate 22 so closely matching the cam 64, it must be understood that the eccentricity of the cam 64 causes the bearing plate 22 to translate relative to the joint body 12 during the throw of the handle 24. The lowest point of the bearing plate curve 66 stays directly under the center of the cam curvature and therefore changes its horizontal offset relative to the handle pivot pin 26 as the handle 24 is thrown.

The bottom surface of the bearing plate 22 is planar against a planar top surface 68 of the lower leg 62 of the joint body 12. Thus, the translation of the bearing plate 22 during the handle throw is achieved by sliding of the bearing plate 22 across the planar top surface 68 of the lower leg 62. The planar surfaces of the bearing plate 22 and the lower leg 62 spread the bearing force against a considerably larger contact area than if a cylindrical or curved cam 64 bore on a planar surface. With the preferred bearing plate 22 formed of a lubricious material, the horizontal sliding of the bearing plate 22 is achieved with minimal friction.

With the bearing plate 22 sliding across a planar surface 68, the bearing plate 22 is not affixed or attached to the lower leg 62. Instead, the bearing plate 22 is captivated by the cam curvature riding in the inner curved surface 66 of the bearing plate 22. The cam 64 is preferably formed with two separate lobes 70, and the bearing plate 22 includes an extending portion 72 which projects between the two lobes 70 of the cam 64. The extending portion 72 prevents the bearing plate 22 from translating sideways relative to the cam 64, keeping the bearing plate 22 in place directly underneath the cam 64. Thus, assembly of the handle 24 and bearing plate 22 into the joint body 12 is quickly and easily achieved merely by positioning the cam 64 of the handle 24 in place in the bearing plate 22, sliding both the handle 24 and bearing plate 22 into position with the pivot pin hole 74 in the handle 24 aligning with the pivot pin hole 76 in the joint body 12, and affixing the handle pivot pin 26 to hold the handle 24 to the joint body 12.

The tightening throw of the handle 24 is preferably relatively short, such as less than about 90°, and more preferably about 45°. To prevent over-tightening or over-loosening of the handle 24 with this short throw, the preferred handle 24 has a loosened stop 78 and a tightened stop 80. The loosened stop 78 restricts a handle 24 from moving past the loosened position by contacting a loosened abutment section 82 of the upper leg 60. The tightened stop 80 restricts the handle 24 from moving past the tightened position by contacting a tightened abutment section 84 of the upper leg 60. Because the loosened stop 78 and the tightened stop 80 both make contact with the upper leg 60 at the end of a throw, a user can tactilely sense when the end of the throw is reached and will not over-torque the clamp 10 beyond the tightened and loosened positions.

The way in which the joint 10 attaches to a support structure is not central to the present invention, and could be affixed in numerous different ways known in the art. Being designed to work with existing Bookwalter/Codman frames, the preferred jaw 20 operates as taught in U.S. patent application Ser. No. 12/037,820 filed Feb. 26, 2008, incorporated by reference, or otherwise known. In general terms, a lower clamping location 86 attaches about the rectangular cross-sectioned stock of a Bookwalter/Codman frame (not shown). The lower clamping location 86 is provided on the top side by the lower leg 62 of the joint body 12 and on the bottom side by the lower jaw 20. On the opposite side of the opening 86, the lower jaw 20 includes a lipped end 88 to clip around the Bookwalter/Codman type frame stock. The lower jaw 20 pivots relative to the joint body 12 about a jaw connection pin 28. A compression spring 30 is housed between the lower jaw 20 and the joint body 12. In the preferred embodiment, the compression spring 30 can be compressed to deflect the lipped end 88 beneath the bottom surface of the Bookwalter/Codman type frame stock with only a few pounds of force.

While the preferred joint 10 does not use the tightening force of the handle 24 to tighten on such Bookwalter/Codman rectangular bar stock, modifications could easily be made such that the force of the cam 64 operates on the lower clamping location 86 as well as tightening on the ball 18. Other clamp body styles may clamp about rods other than Bookwalter/Codman frame stock and other than Bookwalter/Codman blade shafts while fully utilizing the joint 10 of the present invention.

Although the present invention has been described with reference to preferred embodiments, workers skilled in the art will recognize that changes may be made in form and detail without departing from the spirit and scope of the invention.

1. A tightenable ball-and-socket joint for use in a surgical retractor system, comprising:
a socket body fabricated from a unitary structure, the socket body comprising:
a first leg portion having a first compression area which defines a first portion of a generally spherical socket, the first leg portion being relatively inflexible under a tightening force;
a second leg portion having a second compression area, the second compression area defining a second portion of the generally spherical socket which opposes the first compression area, the second leg portion being relatively inflexible under a tightening force; and
a hinge portion between the first leg portion and the second leg portion, the hinge portion being relatively more flexible than the first leg portion and the second leg portion; and
a tightening structure for biasing the first leg portion relative to the second leg portion to press the first compression area toward the second compression area, the tightening structure having a tightened position providing the tightening force and a loosened position; and
a ball member comprising:
a ball received in the generally spherical socket of the socket body, the ball providing a spherical outer profile located between the first compression area and the second compression area and receiving compression force from the first compression area and the second compression area; and
a ball shaft extending from the ball, with the ball shaft being movable in at least one of pitch, yaw and roll relative to the socket body when the tightening structure is in the loosened position, the ball shaft being frictionally restricted in pitch, in yaw and in roll relative to the socket body when the tightening structure is in the tightened position.

2. The tightenable ball-and-socket joint of claim 1, wherein the ball comprises:
a first recess from its spherical outer profile, the first recess corresponding a first leg capture extension of the first leg portion to permit insertion of the ball into the generally spherical socket by alignment of the first recess with the first leg capture extension; and
an interference extension affixed to the ball after the ball has been inserted into the generally spherical socket, the interference extension positioned relative to the recess to prevent removal of the ball from the generally spherical socket even while the tightening structure is in the loosened position.

3. The tightenable ball-and-socket joint of claim 2, wherein the interference extension is a pin.

4. The tightenable ball-and-socket joint of claim 2, wherein the interference extension extends beyond the radius of the spherical outer profile, such that the interference extension limits a range of motion of the ball shaft in at least one of pitch, yaw and roll through interference between the interference extension and the first and second compression areas.

5. The tightenable ball-and-socket joint of claim 2, wherein the ball further comprises:
a second recess from its spherical outer profile, the second recess corresponding to a second leg capture extension of the second leg portion to permit insertion of the ball into the generally spherical socket by alignment of the second recess with the second leg capture extension;
wherein the first recess and the second recess are parallel flats on opposing sides of the ball.

6. The tightenable ball-and-socket joint of claim 1, wherein the tightening mechanism comprises a handle-actuated eccentric cam.

7. The tightenable ball-and-socket joint of claim 6, wherein the cam bears against a bearing plate, with the bearing plate sliding relative to the socket body during movement of the handle-actuated eccentric cam relative to the socket body and the bearing plate.

8. The tightenable ball-and-socket joint of claim 7, wherein the handle-actuated eccentric cam has a lobe which is cylindrical, and wherein the bearing plate has a cylindrical bearing profile against the cylindrical lobe of the handle-actuated eccentric cam.

9. The tightenable ball-and-socket joint of claim 7, wherein the handle-actuated eccentric cam has a plurality of lobes, and wherein the bearing plate has an extension received between the plurality of lobes to generally retain the bearing plate in position relative to the handle-actuated eccentric cam.

10. The tightenable ball-and-socket joint of claim 6, wherein the handle-actuated eccentric cam comprises a loosened stop which restricts a handle from moving past the loosened position and a tightened stop which restricts the handle from moving past the tightened position.

11. The tightenable ball-and-socket joint of claim 1, wherein the first compression area and the second compression area in combination wrap around the ball with a wrap angle of between 190° and 270°.

12. The tightenable ball-and-socket joint of claim 1, wherein the hinge portion is a fulcrum portion adjacent the socket, wherein the first compression area is on a socket side of the fulcrum portion and the first leg portion further comprises a first leg lever arm extending from the fulcrum portion away from the socket, wherein the second compression area is on the socket side of the fulcrum portion and the second leg portion further comprises a second leg lever arm extending from the fulcrum portion away from the socket, and wherein the tightening structure tightens by pushing the first leg lever arm and the second leg lever arm apart.

13. The tightenable ball-and-socket joint of claim 1, wherein the first compression area has a contact profile in contact with the ball which by itself defines a portion of a sphere, and wherein the second compression area has a contact profile in contact with the ball which by itself defines a portion of a sphere.

14. The tightenable ball-and-socket joint of claim 1, wherein the first compression area comprises at least two circumferentially separated contact locations.

15. A method of manufacturing a tightenable ball-and-socket joint, comprising:
fabricating a socket body comprising:
a first leg portion having a first compression area which defines a first portion of a generally spherical socket, the first leg portion having a first leg capture extension;
a second leg portion having a second compression area which opposes the first compression area, the second compression area defining a second portion of the generally spherical socket; and
a connection between the first leg portion and the second leg portion which permits movement between the first compression area and the second compression area;
attaching a tightening structure relative to the first leg portion and the second leg portion such that the tightening structure has a tightened position wherein the first compression area is pressed toward the second compression area, and a loosened position;
aligning a recess of a ball member relative to the first leg capture extension, the ball member comprising:
a ball providing a spherical outer profile size to mate with the first compression area and the second compression area; and
a ball shaft extending from the ball;
inserting the ball into the generally spherical socket during alignment of the first recess with the first leg capture extension; and
affixing an interference extension to the ball positioned relative to the recess to prevent removal of the ball from the generally spherical socket even while the tightening structure is in the loosened position, with the ball shaft being movable in pitch, in yaw and in roll relative to the socket body when the tightening structure is in the loosened position.

16. A tightenable joint for use in a surgical retractor system, comprising:
a joint comprising:
a first leg portion having a first compression area which defines a first portion of a clamp opening; and
a second leg portion having a second compression area which opposes the first compression area, the second compression area defining a second portion of the clamp opening; and
a connection between the first leg portion and the second leg portion which permits movement between the first compression area and the second compression area;
a tightening structure for biasing the first leg portion relative to the second leg portion to press the first compression area toward the second compression area, the tightening structure having a tightened position and a loosened position, the tightening structure comprising:
a handle-actuated eccentric cam, and
a bearing plate which the handle-actuated eccentric cam bears against, with the bearing plate sliding relative to the socket body during movement of the handle-actuated eccentric cam relative to the joint body and the bearing plate; and
a received component sized to be received in the clamp opening such that the received component is moveable within the clamp opening when the tightening structure is in the loosened position but secured within the clamp opening when the tightening structure is in the tightened position.

17. The tightenable joint of claim 16, wherein the handle-actuated eccentric cam has a lobe which is cylindrical, and wherein the bearing plate has a cylindrical bearing profile against the cylindrical lobe of the handle-actuated eccentric cam.

18. The tightenable joint of claim 16, wherein the handle-actuated eccentric cam has a plurality of lobes, and wherein the bearing plate has an extension received between the plurality of lobes to generally retain the bearing plate in position relative to the handle-actuated eccentric cam.
